(19) United States
(12) Patent Application Publication
(10) Pub. No.: US 2020/0116716 A1
(43) Pub. Date: Apr. 16, 2020

(54) DETECTION OF LEUKOCYTE-DERIVED MICROVESICLES BY FLUO-SENSITIVE FLOW CYTOMETRY

(71) Applicant: Beckman Coulter, Inc., Brea, CA (US)

(72) Inventors: Romaric Lacroix, Marseille (FR); Stephane Robert, Marseille (FR); Francoise Dignat George, Marseille (FR); Emmanuel Gautherot, Marseille (FR)

(21) Appl. No.: 16/621,921

(22) PCT Filed: Jun. 13, 2018
(86) PCT No.: PCT/US2018/037374

§ 371 Date: Dec. 12, 2019

Related U.S. Application Data
(60) Provisional application No. 62/519,059, filed on Jun. 13, 2017.

(51) Int. Cl. G01N 33/566; G01N 1/30; G01N 1/34; G01N 15/14

(52) U.S. Cl. CPC G01N 33/566; G01N 1/30; G01N 1/34; G01N 15/1404; G01N 2015/1006; G01N 2001/302; G01N 2333/70539; G01N 2333/70553; G01N 2333/70596; G01N 15/1456

(57) ABSTRACT

This invention provides methods for characterizing microvesicles using a cocktail of labeled antibodies. The invention also provides kits that can be used for this purpose.

### RELATED APPLICATIONS

This application claims the benefit of U.S. Provisional Application No. 62/519,059, filed on Jun. 13, 2017. The entire content of said provisional application is hereby incorporated by references for all purposes.

### FIELD OF THE INVENTION

This invention relates to compositions and methods for detecting and characterizing microvesicles.

### BACKGROUND OF THE INVENTION

Leukocyte-derived microvesicles (LMV) have been identified as an important biomarker for a variety of diseases, e.g., cardio-vascular diseases, inflammation, and sepsis. Accurate and timely detection of LMV is thus important for cardio-vascular disease diagnosis, prevention, and treatment. Flow cytometry is a useful tool for detecting cellular structures. However, this approach has been shown to be difficult in detecting LMVs because of their scarcity, small size, and low antigen density. The current flow cytometry methods are also limited by instrument sensitivity and the high background noise.

### BRIEF SUMMARY OF THE INVENTION

This invention provides methods and kits for characterizing microvesicles. In one aspect, the disclosure provides a method for characterizing microvesicles comprising staining a sample including a microvesicle with a cocktail to form a stained microvesicle; measuring the stained microvesicle by flow cytometry to obtain a signal set; and identifying the stained microvesicle as a leukocyte-derived microvesicle when the signal set of the measured microvesicle is within a signal region.

In another aspect, the disclosure provides a kit comprising a cocktail including labeled antibodies to at least one of HLA DR, CD11c, CD66c, CD18, and CD157. Also provided is a kit comprising a cocktail including at least one of a first group of labeled antibodies to HLA DR and CD11c, a second group of labeled antibodies to CD66c, a third group of labeled antibodies to CD18 and CD157, and a fourth group of labeled antibodies to TIA-1.

Further features and advantages of the invention will become more fully apparent in the following description of the embodiments and drawings thereof, and from the claims.

### BRIEF DESCRIPTION OF THE DRAWINGS

FIG. 1 illustrates the procedures of production and purification of microvesicles (MV).

FIGS. 2A-2C show the results from flow cytometry analysis of samples comprising LMVs. Samples were stained with PE-labeled HLA DR antibody and FITC-labeled annexin V (FIG. 2A); PE-labeled CD15 antibody and FITC-labeled Annexin V (FIG. 2B); or PE-labeled CD18 antibody and FITC-labeled Annexin V (FIG. 2A).

FIG. 3A shows detecting LMVs in samples that were serially-diluted with plasma. The samples were stained with PE-labeled CD15 antibody and FITC-labeled Annexin V. FIG. 3B shows graphs in which the amount of double positive LMVs was plotted against the dilution factor for each sample.

FIG. 4A shows detecting LMVs using methods involving washing stained LMVs by size exclusion chromatography in order to remove unbound antibodies. FIG. 4B shows improvement on reduction of background noise by introducing the wash step on samples stained with each one of the different antibodies, i.e., HLA-DR, CD45, CD66c, CD14, CD31, CD18, CD157, CD11C, and CD15 antibodies.

FIG. 5A shows the results of detecting LMVs using a panel of PE-labeled antibodies, the CD 18, CD147, and CD45 antibodies, to stain LMVs in the same assay. The assay was performed under no wash conditions, i.e. no size exclusion chromatography was used to remove the unbound antibodies before flow cytometry analysis. FIG. 5B shows that the three-antibody combination detected higher amount of LMVs than any of the three antibodies used alone and FIG. 5C shows that the mean fluorescent intensity (MFI) from the assays using the three-antibody combination was also slightly higher than the MFI from the assays using any individual antibody alone.

FIGS. 6A and 6B show the results from assays performed under conditions identical to those in FIGS. 5A and 5B, except that they included a washing step using size exclusion chromatography to remove unbound antibodies before the flow cytometry analysis. Similar to the results in FIGS. 5A and 5B, the three-antibody combination detected more LMVs than any of the three antibodies used alone. FIG. 6C shows that the three-antibody combination also increased MFI significantly than any of the three antibodies used alone.

FIGS. 7A-7C compare results of LMV counts from the same samples analyzed using Gallios versus CytoFLEX. In addition to being stained with FITC-labeled Annexin V, samples were also stained with a PE-labeled HLA DR antibody (FIG. 7A), or a PE-labeled CD11c (FIG. 7B). FIG. 7C shows counts of MV detected by CytoFLEX were significantly higher than counts of MVs detected by Gallios.

### DETAILED DESCRIPTION OF THE INVENTION

### 1. Overview

This invention provides compositions, kits, and methods using one or more antibodies in flow cytometry analysis to detect LMVs in biological samples with desired sensitivity and specificity. In some embodiments, the disclosed compositions, kits and methods utilize unique panels of antibodies, washing steps, and/or instruments that are ultra-sensitive at detecting fluorescence to detect the LMVs.

### 2. Definitions

As used herein, “a labeled molecule” refer to a molecule that is attached to a detectable label, which can be identified in flow cytometry. The attachment between the molecule and the detectable label can be direct or indirect. In preferred embodiments, the attachment between the molecule and the detectable label is formed by direct conjugation.

As used herein, the term “a signal region” refers to a region in a flow cytometry plot, which the signal from a MV that expresses a marker of interest would fall within. In some cases, the marker is a molecule (e.g., an antigen) on the MV that can be recognized by a component (e.g., an antibody) of the cocktail used to stain the MV. One skilled in the art of flow cytometry can readily determine the signal region for each marker, e.g., by comparing the results from a positive control, i.e. a sample that is known to express the marker, with the result from a negative control, i.e., a unstained sample, or a sample that is known to not express the marker.

As used herein, the term “positive” refers to a signal indicating the presence of a marker of interest in the MV.

As used herein, the term “a signal set” refers to a set of signals produced by components of the cocktail that bind to the MVs in a sample.

The term “antibody” includes monoclonal antibodies, polyclonal antibodies, synthetic antibodies and chimeric antibodies, e.g., generated by combinatorial mutagenesis and phage display. The term “antibody” also includes mimetics or peptidomimetics of antibodies. Peptidomimetics are compounds based on, or derived from, peptides and proteins.

The peptidomimetics of the present invention typically can be obtained by structural modification of a known peptide sequence using unnatural amino acids, conformational restraints, isosteric replacement, and the like. Fragments of antibodies may serve in place of antibodies in some embodiments.

### 3. Specific Embodiments

### Samples

The invention may be used to characterize the origin of microvesicles in a sample. Samples to be assayed for the presence of LMVs by the methods of the present invention include, for example, human and animal body fluids such as whole blood, serum, plasma, cerebrospinal fluid, sputum, bronchial washings, bronchial aspirates, urine, lymph fluids and various external secretions of the respiratory, intestinal and genitourinary tracts, tears, saliva, milk, white blood cells, myelomas and the like; biological fluids such as cell culture supernatants; tissue specimens; pleural fluid, or homogenates.

### Leukocytes

Leukocytes, commonly referred to as white blood cells, are typically divided into several classes based on morphological and tinctorial characteristics when stained: granulocytes (including neutrophils, eosinophils, and basophils), monocytes, and lymphocytes. Both granulocytes and monocytes belong to the category of myeloid cells.

### Microvesicles (MVs)

MVs are fragments of plasma membrane ranging from 100 nm to 1000 nm, which are shed from plasma membrane from resting or stimulated cells. They function to transport proteins, RNA and other molecules that contain biological information. They can also remove misfolded proteins, cytotoxic agents and metabolic waste from the cell and play important roles in intercellular communication.

Leukocyte-derived MVs are MVs shed from the plasma membranes of leukocytes. In some embodiments, the LMVs are derived from myeloid cells, e.g., from granulocytes or monocytes. In some embodiments, the LMVs are derived from leukocytes under a basal condition or a stimulated condition. LMVs reflect the antigenic content of the leukocytes from which they originate and can be potential biomarkers, especially for patients with atherosclerosis, diabetes, or sepsis. Thus, detecting and characterizing LMVs that are derived from specific subtypes of leukocytes and specific conditions have significant clinical implications.

### Cocktail

Disclosed herein is a cocktail that can be used to detect LMVs. The cocktail include one or more of HLA DR antibody, CD11c antibody, CD18 antibody, CD157 antibody, Annexin V, and TIA-1 antibody, each labeled with a label (as discussed below) that can be detected in flow cytometry. In some embodiments, the cocktails include a first group, a second group, and/or a third group. The first group comprises an antibody to HLA DR and an antibody to CD11c, each labeled with a first label; the second group comprises an antibody to CD66c, labeled with a second label; and a third group comprises an antibody to CD18 and an antibody to CD157. In some embodiments, the second group further comprises labeled antibodies to CD15. In some embodiments, the third group further comprises labeled antibodies to CD45. In some embodiments, the cocktail further includes annexin V labeled with a fourth label. Annexin V recognizes phosphatidylserine (PS). In intact cells, PS is on the inner leaflet of the plasma membrane. Microvesicles that are shed from intact cells and some of which have PS exposed and accessible to annexin V. Thus, annexin V can be used to distinguish all relevant MVs from non-specifically stained particles, unactivated platelets, and debris. TIA-1 is a marker present on the surface of stimulated cells. In some embodiments, the cocktail used to detect LMVs further include antibodies to TIA-1 labeled with a fifth label.

In some cases, the method of detecting LMVs involves staining the sample with a cocktail comprising at least one antibody from the first group and the labeled annexin V, provided that the first label (used to label the first group of antibodies) is distinguishable from the fourth label (used to label annexin V). The LMV is assigned as monocyte-derived if it is positive for the marker recognized by the at least one antibody from the first group and the marker recognized by annexin V. In some embodiments, the first group of labeled antibodies comprise HLA-DR, CD44, CD31, CD45, CD11b, CD157, and CD18 antibodies. In some embodiments, the at least one antibody from the first group is HLA DR and/or CD11c antibodies.

In some cases, the method involves staining the sample with a cocktail comprising at least one antibody from the second group of labeled antibodies and the labeled annexin V, provided that the second label (used to label the second group of antibodies) and the fourth label (used to label annexin V) are distinguishable. The LMV is assigned as granulocyte-derived if it is positive for the marker recognized by the at least one antibody from the second group and the marker recognized by annexin V. In some embodiments, the at least one antibody is selected from the second group is CD15 and/or CD66c antibodies. In some embodiments, the second group comprises CD15, CD18, CD24, CD66c, CD59, CD157, CD66b, CD11b, CD45, CD65, and lactoferrin antibodies.

In some cases, the method involves staining the sample with a cocktail comprising at least one antibody from the third group of labeled antibodies and the labeled annexin V, provided that the third label (used to label the third group of antibodies) and the fourth label (used to label annexin V) are distinguishable. The LMV is assigned as myeloid-derived if it is positive for the marker recognized by the at least one antibody from the third group and the marker recognized by annexin V. In some embodiments, the third group comprises CD18, CD157, and CD45 antibodies.

In some cases, the method involves staining the sample with a cocktail comprising a labeled TIA-1 antibody and the labeled annexin V, provided that the fifth label (used to label TIA-1) and the fourth label (used to label annexin V) are distinguishable. The LMV is assigned as derived from an activated cell if it is positive for the marker recognized by TIA-1 and the marker recognized by annexin V.

In some cases, the method involves staining the sample comprising LMVs with a cocktail comprising a labeled annexin V and antibodies from two or more of i) the first group, ii) the second group, iii) the third groups, and iv) the TIA-1 antibody, provided the labels on the antibodies used for the staining are distinguishable from one another. For example, a method may involve staining the sample with at least one antibody from the first group, one TIA-1 antibody, and annexin V, each being labeled with a label that distinguishable from one another; a LMV may be assigned as derived from an active monocyte if it is positive for all three markers.

### Labels

The labels used in the invention can be any label that can be detected by flow cytometry. Non-limiting examples of the labels include those listed in Table 1, below.

TABLE 1
Some exemplary labels used to label antibodies.

| | | Absorption | Emission |
|---|---|---|---|
| Fluorochrome | Abbreviation | max. nm | max. nm |
| violet laser diode | | | |
| Cascade blue | | 380-401 | 419 |
| Cascade yellow | | 399 | 549 |
| Pacific Blue | | 405 | 456 |
| argon-ion laser | | | |
| fluorescein | FITC | 495 | 520 |
| Alexa 488 | | 488 | 520 |
| R-phycoerythin | PE | 495 564 | 576 |
| PE-Texas Red | ECD | 495 564 | 620 |
| PE-cyanine 5 | PC5 | 495 564 | 670 |
| Peridinin-chlorophyll | PerCP | 490 | 677 |
| PE-cyanine-5.5 | PC5.5 | 495 564 | 696 |
| PE-cyanine 7 | PC7 | 495 564 | 767 |
| He—Ne laser | | | |
| allophycocyanin | APC | 650 | 660 |
| APC-cyanine 5.5 | APC-Cy5.5 | 650 | 710 |
| APC-cyanine 7 | APC-Cy7 | 650 | 767 |

Other labels that can be used to label the antibodies include Quantum dots. Quantum dots are fluorescent nanocrystals that have a wide absorption spectrum and so can be excited by a range of different wavelengths. Their emission wavelengths also vary and may range from blue to deep red. One skilled in the art can readily determine which types of labels to use based on the purpose of the assay. In some embodiments, at least one of the first, second, third label, which are used to label the antibodies, is PE, and the fourth label, which is used to label annexin V, is FITC.

### Methods

In some embodiments, the present disclosure provides a method of characterizing MVs, the method comprising staining a sample including a MV with a cocktail to form a stained MV, measuring the stained MVs and identifying the stained microvesicle as a leukocyte-derived microvesicle when the signal set of the measured microvesicle is within a signal region. The cocktail used to stain the sample may comprise one or more of the labeled antibodies and/or a labeled annexin V, as described above. In one embodiment, the measuring the stained MVs is by flow cytometry.

In some embodiments, samples comprising MVs are prepared by separating whole blood using a ficoll gradient method, followed by ultrafiltration to concentrate MVs. In some embodiments, a size exclusion chromatography is also used to purify MVs after the ultrafiltration.

In some embodiments, staining the sample can be performed by adding the labeled antibodies and/or annexin V to the sample and incubated the mixture for a period of time, typically between 20 minutes and 1 hour. The incubation are typically performed at room temperature, for example, for 18° C. to 25° C., e.g., 20° C. to 25° C.

In some embodiments, the method further comprises a step of separating unbound materials, e.g., antibodies, from the stained MVs before measuring the signals from the stained MVs. The separating unbound material may include washing the stained mixture on a size exclusion chromatography column with a calcium containing buffer. Suitable size exclusion chromatography (SEC) column that can be used for the separation include qEV, which are commercially available from Izon. In some embodiments, the amount of calcium in the calcium-containing buffer may range from 0.5 to 2.5 mM, e.g., from 1 to 2 mM. In one embodiment, the calcium-containing buffer is annexin V binding buffer, which can be readily obtained from various commercial sources. For preparation of the In some embodiments, the SEC column is loaded with a sepharose matrix and an annexin V binding buffer. The stained MVs are then loaded to the column to separate the unbound materials. The stained MVs are then eluted from the column using an elution buffer. Preferably, the elution buffer contains calcium. In one embodiment, the elution buffer is an annexin binding buffer.

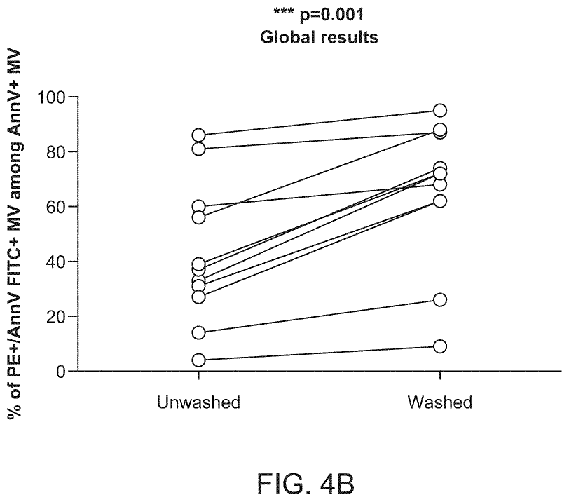

Washing the stained mixture on a size exclusion chromatography column as described above can dramatically decrease the fluorescent background noise and thus more stained LMVs, including those produce low signals, can be detected. The degree of improvement may vary depending on the type of antibody used for staining as well as the concentration of the antibody. In some embodiments, including the step of washing the stained mixture on a SEC column before flow cytometry analysis can increase the counts of the detected LMVs by between 10% and 330%, e.g., between 50% to 330%, between 90% to 200%, or between 170% and 210%. In one particular embodiment, as shown in FIG. 4A, the method comprises staining the sample with a labeled CD45 antibody and a labeled annexin V and separating the unbound material by washing the stained mixture on a SEC column; the counts of myeloid-derived LMVs detected increased about 150% as compared to a method that does not use a wash step using the SEC. In another particular embodiment, as shown in FIG. 4B, the method uses a labeled HLA DR and a labeled annexin V to stain a sample and separating the unbound material by washing the stained mixture on a SEC column; the counts of the detected monocyte-derived LMVs increased about 100% as compared to the method that did not use the wash step.

### Flow Cytometry

Flow cytometry can be used to measure and characterize cells and structures, e.g., MVs in a fluid as they pass through one or multiple lasers insider a flow cytometer. Labeled MVs emit light of different wavelengths upon excitation by a specific laser and the emission. The flow cytometer record and/or analyze relative fluorescence, cell size, and relative granularity, and thus can be especially useful for biomarker detection, e.g., the markers on the MVs.

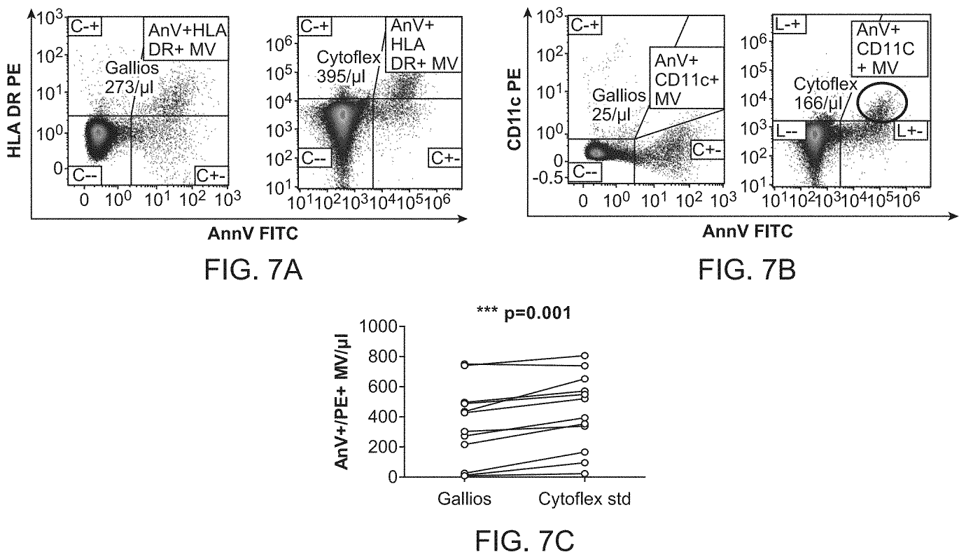

The methods and kits disclosed herein can be used in any flow cytometers to detect the stained LMVs. Non-limiting examples of flow cytometers include Gallios, CytoFLEX, and Navios. Due to the rarity of the LMVs and low density of antigenic sites on the LMVs, flow cytometers that have high fluorescence detection sensitivity, e.g., CytoFLEX from Beckman Coulter, is preferred. As shown in FIGS. 7A-7C, a significant increase in LMVs counts was observed when detected using cytoFLEX as compared to Gallios.

### Kits

The present invention also provides a kit comprising a cocktail including labeled antibodies to at least one of HLA DR, CD11c, CD66c, CD18, and CD157. In some embodiments, the kit comprising a cocktail including at least one of: i) a first group of labeled antibodies to HLA DR and CD11c; ii) a second group of labeled antibodies to CD66c; iii) a third group of labeled antibodies to CD18 and CD157; and iv) fourth group of labeled antibodies to TIA-1. In some embodiments, the second group further includes labeled antibodies to CD15, and wherein the third group further includes labeled antibodies to CD45. In some embodiments, each of the antibodies in the first group is labeled with a first label, wherein each of the antibodies in the second group is labeled with a second label, wherein each of the antibodies in the third group is labeled with a third label, and wherein each of the antibodies in the fourth group is labeled with a fourth label. In some embodiments, the cocktail further includes annexin V labeled with a fifth label.

In some embodiments, the kit further comprises an elution buffer and/or instructions on how to use the kit.

### Systems

This invention also provides a system comprising a component for staining a sample including a microvesicle with a cocktail to form a stained microvesicle, a component for measuring the stained microvesicle, and a component for identifying the stained microvesicle as for leukocyte-derived microvesicle when the signal set of the measured microvesicle is within a signal region. In some embodiments, the component for measuring the stained microvesicle includes a flow cytometer. In some embodiments, one or more of the components of the system includes one or more computer processors, which execute instructions to carry out the steps of any of the methods of characterizing microvesicles as disclosed above.

### Embodiments

The following are some exemplary embodiments of the disclosure.

1. A method of characterizing microvesicles comprising:
staining a sample including a microvesicle with a cocktail to form a stained microvesicle;
measuring the stained microvesicle by flow cytometry to obtain a signal set; and identifying the stained microvesicle as a leukocyte-derived microvesicle when the signal set of the measured microvesicle is within a signal region.
   2. The method of embodiment 1, wherein the cocktail includes labeled antibodies to at least one of HLA DR, CD11c, CD66c, CD18, and CD157.
   3. The method of embodiment 2, wherein the cocktail comprises labeled annexin V.
   4. The method of embodiment 2 or 3, wherein the cocktail comprises at least one of
   i) a first group of labeled antibodies comprising an antibody to HLA DR and an antibody to CD11c, each labeled with a first label;
   ii) a second group of labeled antibodies comprising an antibody to CD66c, labeled with a second label;
   iii) a third group of labeled antibodies comprising an antibody to CD18 and an antibody to CD157, each labeled with a third label.
   5. The method of embodiment 4, wherein second group further includes labeled antibodies to CD15, and wherein the third group further includes labeled antibodies to CD45.
   6. The method of embodiment 4, wherein the labeled annexin V is labeled with a fourth label.
   7. The method of embodiment 5 or 6, wherein the cocktail further includes antibodies to TIA-1 labeled with a fifth label.
   8. The method of embodiment 5 or 6, wherein the step of identifying includes assigning the microvesicle as monocyte-derived when the signal set indicates binding of at least one antibody of the first group.
   9. The method of embodiment 5 or 6, wherein the step of identifying includes assigning the microvesicle as granulocyte-derived when the signal set indicates binding of least one antibody of the second group.
   10. The method of embodiment 5 or 6, wherein the step of identifying includes assigning the microvesicle as myeloid-derived when the signal set indicates binding of least one antibody of the third group.
   11. The method of embodiment 7, wherein the step of identifying includes assigning the microvesicle as derived from an activated cell when the signal set indicates binding of antibody to TIA-1.
   12. The method of embodiment 1, further including separating unbound material from the stained microvesicle.
   13. The method of embodiment 12, wherein the step of separating unbound material includes washing the stained mixture on a size exclusion chromatography column loaded with a calcium-containing buffer.
   14. A kit comprising a cocktail including labeled antibodies to at least one of HLA DR, CD11c, CD66c, CD18, and CD157.
   15. A kit comprising a cocktail including at least one of:
a first group of labeled antibodies to HLA DR and CD11c;
a second group of labeled antibodies to CD66c;
a third group of labeled antibodies to CD18 and CD157; and a fourth group of labeled antibodies to TIA-1.
   16. The kit of embodiment 15, wherein the second group further includes labeled antibodies to CD15, and wherein the third group further includes labeled antibodies to CD45.
   17. The kit of embodiment 15 or 16, wherein each of the antibodies in the first group is labeled with a first label, wherein each of the antibodies in the second group is labeled with a second label, wherein each of the antibodies in the third group is labeled with a third label, and wherein each of the antibodies in the fourth group is labeled with a fourth label.
   18. The kit of any of embodiments 14-17, wherein the cocktail further includes annexin V labeled with a fifth label.
   19. The kit of any of embodiments 14-18, further comprising an elution buffer.
   20. A system for characterizing microvesicles comprising one or more processors and memory coupled to one or more processors, the memory encoded with a set of instructions configured to perform a method comprising:
obtaining measurements of a microvesicle stained with a cocktail from a flow cytometer, and
identifying the stained microvesicle as leukocyte-derived microvesicle when the signal set of the measured microvesicle is within a signal region.
   21. A system for characterizing microvesicles comprising one or more processors and memory coupled to one or more processors, the memory encoded with a set of instructions configured to perform one or more method steps of the method of any of embodiments 1-13.

### IV. EXAMPLES

The present invention is described by reference to the following Examples, which are offered by way of illustration and are not intended to limit the invention in any manner. Standard techniques well known in the art or the techniques specifically described below were utilized.

### Example 1. Methods

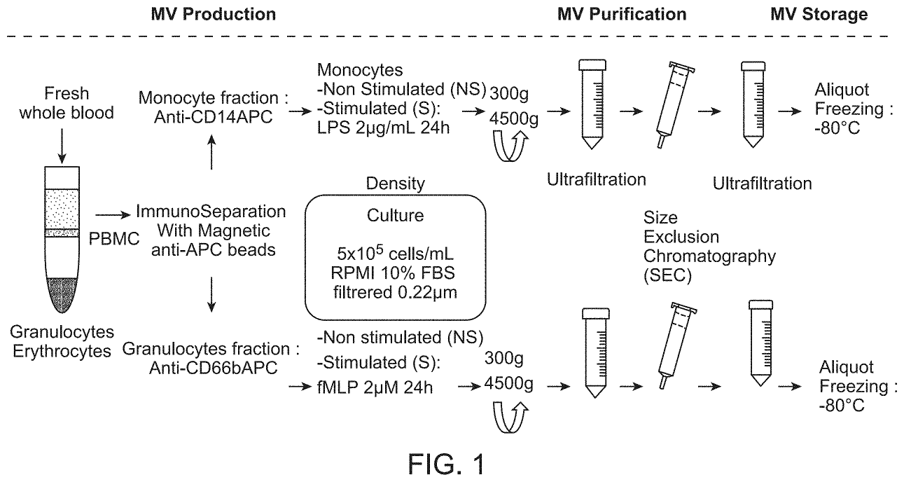

The procedures for producing and purifying monocytes and granulocytes microvesicles are illustrated in FIG. 1. In brief, fresh whole blood was separated by ficoll gradient and then by immune-magnetic separation to extract monocytes and granulocytes. The monocytes and granulocytes obtained using the method had a purity of greater than 94%. Cells were then incubate during a 24-hour time period in media supplemented with LPS to produce monocytes and fMLP to produce granulocytes. Supernatants from the cell culture were then collected, and after serial centrifugations, MVs were purified using ultrafiltration to concentrate MVs and size exclusion chromatography to purify MVs. The obtained MVs were re-suspended in PBS buffer, aliquoted and freezed at −80° C. for the study.

### Example 2. Antibody Screening on LMV

MVs prepared from Example 1 were used to screen a panel of antibodies. Each of the leucocyte specific antibodies listed in Table 2 was used to stain sample in combination with annexin V. Annexin V can distinguish MVs from intact cells, were used to stain MVs. In this experiment, each antibody is conjugated to PE and annexin V is conjugated to FITC. A titration with 5 different concentrations was performed for all antibodies. To avoid aggregates, antibodies were centrifuged at 13,000 g for 2 minutes prior to use. In addition, a fluorescently-matched isotype control was used for each concentration. Percentages of PE and Annexin V double positive population among the total annexin V positive MV population were calculated for each antibody, and 20% was considered the cut off—if the percentages is 20% or higher, the antibody can be used to detect the MV population.

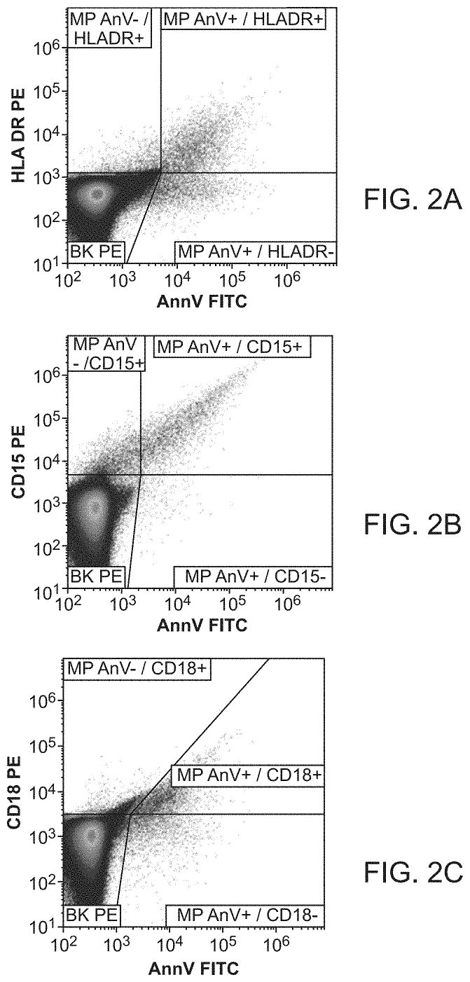

As shown in Table 2, 11 antibodies (i.e., antibodies to CD15, CD18, CD24, CD66c, CD59, CD157, CD66b, CD11b, CD45, CD65, lactoferrin) were shown to meet the criteria for detecting granulocyte-derived MVs and 7 antibodies (i.e., antibody to HLA-DR, CD44, CD31, CD45, CD11b, CD157, CD18) met the criteria for detecting monocyte-derived MVs. The antibodies were ranked based on the percentages of LMVs detected, from high to low. The top-performing antibodies that can be used to detect monocyte-derived MVs are shown in Table 3 and the top-performing antibodies that can be used to detect granulocyte-derived MVs are shown in Table 4. Antibodies that can be used to detect myeloid MVs (i.e., both monocyte-derived and granulocyte-derived MVs) are shown in Table 5. These antibodies are known to recognize specific markers on different subpopulation of the leukocytes; for example, HLA-DR is a marker for monocytes, CD15 is a marker for granulocytes, and CD18 is a marker for both monocytes and granulocytes. FIG. 2A shows a population that is positive for both the HLA DR antibody and Annexin V staining, which correspond to monocyte-derived MVs. FIG. 2B shows a population that is positive for both the CD15 antibody and Annexin V staining, which correspond to granulocyte-derived MVs. FIG. 2C shows a population that is positive for both the CD18 antibody and Annexin V staining, which correspond to myeloid-derived MVs.

TABLE 2
Screening for the antibodies that can be used to detect markers on LMVs

| Markers | Mono NS | Mono S | Gran NS | Gran S |
|---|---|---|---|---|
| CD18 | 49.3 | 57.7 | 52.9 | 47.6 |
| CD45 | 39.1 | 45.0 | 25.4 | 26.7 |
| HLA-DR | 38.6 | 43.0 | 0.8 | 0.6 |
| CD157 | 37.9 | 41.7 | 30.0 | 27.2 |
| CD31 | 27.9 | 24.3 | 11.0 | 12.2 |
| CD44 | 24.7 | 27.8 | 9.7 | 10.3 |
| CD11b | 21.9 | 17.3 | 25.8 | 24.0 |
| CD36 | 17.0 | 16.1 | 5.5 | 5.4 |
| CD11c | 13.6 | 15.2 | 4.0 | 4.3 |
| CD40 | 11.4 | 6.6 | 4.0 | 3.9 |
| CD55 | 9.4 | 9.2 | 8.9 | 8.1 |
| CD33 | 9.2 | 8.9 | 5.5 | 5.4 |
| CD15 | 7.4 | 8.0 | 81.0 | 79.1 |
| CD59 | 6.5 | 6.2 | 41.2 | 37.9 |
| CD32 | 6.5 | 5.3 | 17.9 | 19.6 |
| CD66b | 6.2 | 4.8 | 27.9 | 26.5 |
| CD29 | 6.2 | 7.9 | 1.3 | 1.7 |
| CD65 | 5.7 | 3.9 | 21.7 | 22.7 |
| CD11a | 5.5 | 8.9 | 2.5 | 2.7 |
| CD49d | 4.8 | 4.9 | 7.1 | 5.2 |
| CD13 | 4.7 | 6.2 | 8.9 | 8.4 |
| CD66c | 4.7 | 4.3 | 50.8 | 52.8 |
| CD24 | 4.7 | 5.2 | 52.4 | 48.2 |
| CD69 | 4.1 | 2.4 | 3.0 | 2.3 |
| CD86 | 3.9 | 4.4 | 1.9 | 1.9 |
| CD58 | 3.5 | 3.5 | 2.9 | 4.1 |
| CD85j | 3.5 | 3.7 | 4.6 | 2.8 |
| CD328 | 3.1 | 2.4 | 1.7 | 2.4 |
| CD14 | 2.6 | 2.4 | 1.7 | 1.9 |
| TIA-1 | 2.2 | 1.7 | 12.7 | 23.3 |
| CD16 | 2.2 | 1.5 | 19.0 | 17.3 |
| CD10 | 2.1 | 1.5 | 7.6 | 13.3 |
| CD48 | 2.1 | 2.3 | 1.0 | 0.7 |
| CD85k | 2.0 | 0.9 | 0.4 | 0.6 |
| CD184 | 2.0 | 1.7 | 2.0 | 2.3 |
| CD300a | 1.9 | 2.5 | 1.9 | 2.5 |
| CD116 | 1.9 | 1.8 | 0.7 | 1.2 |
| CD43 | 1.8 | 2.5 | 6.1 | 7.6 |
| CD26 | 1.7 | 1.1 | 2.5 | 1.9 |
| CD126 | 1.5 | 1.3 | 1.9 | 1.1 |
| Lactoferrin | 1.5 | 1.7 | 20.5 | 23.9 |
| CD120b | 1.5 | 1.4 | 2.0 | 1.4 |
| CD64 | 1.4 | 1.0 | 1.2 | 0.9 |
| CD54 | 1.4 | 0.9 | 0.4 | 0.4 |
| CD90 | 1.4 | 1.2 | 0.9 | 1.2 |
| CD85d | 1.3 | 1.1 | 1.7 | 1.5 |
| CD62P | 1.3 | 0.8 | 0.7 | 0.9 |
| CD166 | 1.0 | 0.8 | 1.1 | 0.8 |
| CD106 | 0.9 | 0.3 | 0.5 | 0.3 |
| CD62L | 0.9 | 0.2 | 0.8 | 0.3 |
| OSCAR | 0.7 | 1.0 | 0.9 | 0.9 |
| CD105 | 0.6 | 0.4 | 0.3 | 0.4 |
| CD123 | 0.6 | 0.8 | 1.1 | 1.1 |
| CD122 | 0.5 | 0.6 | 0.5 | 0.5 |
| CD109 | 0.4 | 1.0 | 1.4 | 1.0 |

Note: “Mono” stands for MVs derived from monocytes. “Gran” stands for MVs derived from granulocytes. “NS” stands for MVs derived from cells under non-stimulated or basal condition. “S” stands for MVs derived from cells under stimulated condition. The numerical values in this Tables 2-5 represent the percentages of PE and Annexin V double positive MV population among the total annexin V positive MV population.

TABLE 3
Top-performing antibodies that detect markers on monocyte-derived MVs

| Markers | Mono NS | Mono S | Gran NS | Gran S |
|---|---|---|---|---|
| HLA-DR | 38.6 | 43.0 | 0.8 | 0.6 |
| CD11c | 13.6 | 15.2 | 4.0 | 4.3 |

TABLE 4
Top-performing antibodies that detect markers on
granulocyte-derived MVs

| Markers | Mono NS | Mono S | Gran NS | Gran S |
|---|---|---|---|---|
| CD15 | 7.4 | 8.0 | 81.0 | 79.1 |
| CD66c | 4.7 | 4.3 | 50.8 | 52.8 |

TABLE 5
Exemplar antibodies that detect myeloid-derived MVs

| Markers | Mono NS | Mono S | Gran NS | Gran S |
|---|---|---|---|---|
| CD18 | 49.3 | 57.7 | 52.9 | 47.6 |
| CD157 | 37.9 | 41.7 | 30.0 | 27.2 |
| CD45 | 39 | 45 | 25 | 27 |

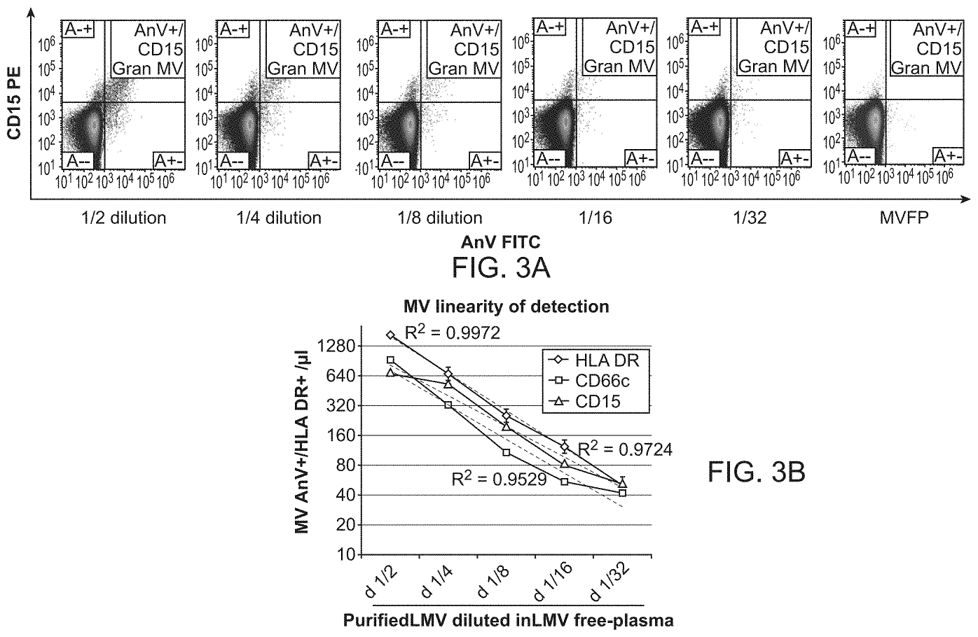

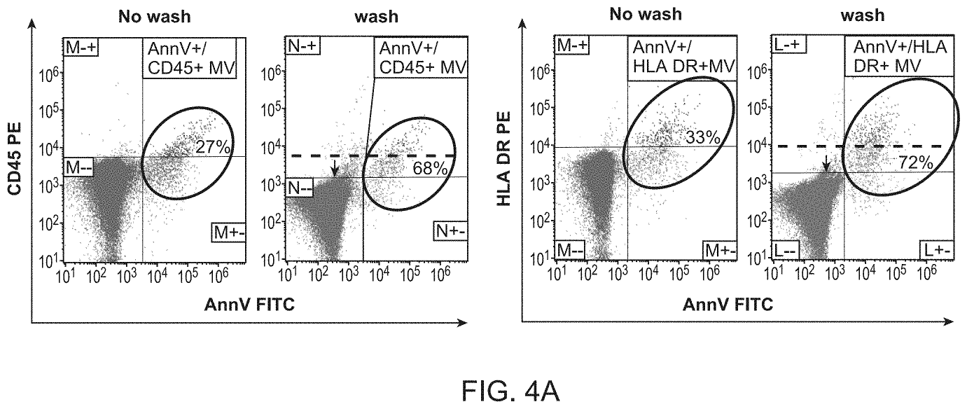

In order to validate the assay and assess its linearity, the MVs obtained using methods described in Example 1 were serially diluted in MV-free plasma, in a range from 1:2 to 1:32. Absolute quantification of MVs was performed using counting beads. The samples comprising MVs were stained with CD15 antibodies labeled with PE and annexin V labeled with FITC and analyzed by flow cytometry (FIG. 3A). Similar experiments were performed with HLA DR and CD66c antibodies. The result showed that the assays using each of the HLA DR, CD66c, and CD15 antibodies, were linear even when the concentrations of the MVs in the sample is in very low, e.g., less than 50 MV/μl, e.g., between 30-50 MV/μl (FIG. 3B).

### Example 3. Size Exclusion Chromatography for Washing Stained MVS

Samples that were labeled with antibodies to specific markers as identified in Example 2 were loaded with an annexin V binding buffer (which contains calcium) to a size exclusion chromatography (SEC) column with a sepharose matrix. The results showed that, in general, washing the stained mixture on the SEC column reduced background fluorescence, as reflected by an increase in the detected MV count and an increase in the mean fluorescence of the signal; the percentage of the MVs detected may increase by 10% to 200% (see FIG. 4B). The degree of increase may vary depending on the inherent properties and concentrations of the antibody used in the staining, as well as the antigen density on the MVs. For example, in one experiment, only a slight reduction of background noise and only a slight gain in the count of double positive MVs were observed for a CD 66c-stained sample that was washed using the SEC column as compared to non-wash samples. In another experiment, the increase in detected LMVs and in MFI caused by the wash step was significant with CD45 or HLA DR-stained samples as compared to similarly stained samples which were not washed (FIG. 4A).

### Example 4. Antibody Combination for LMV Detection

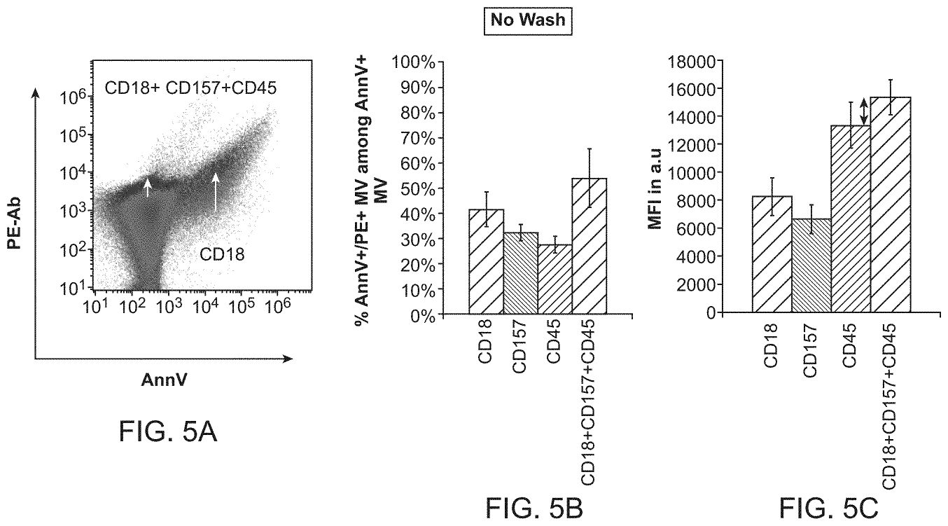

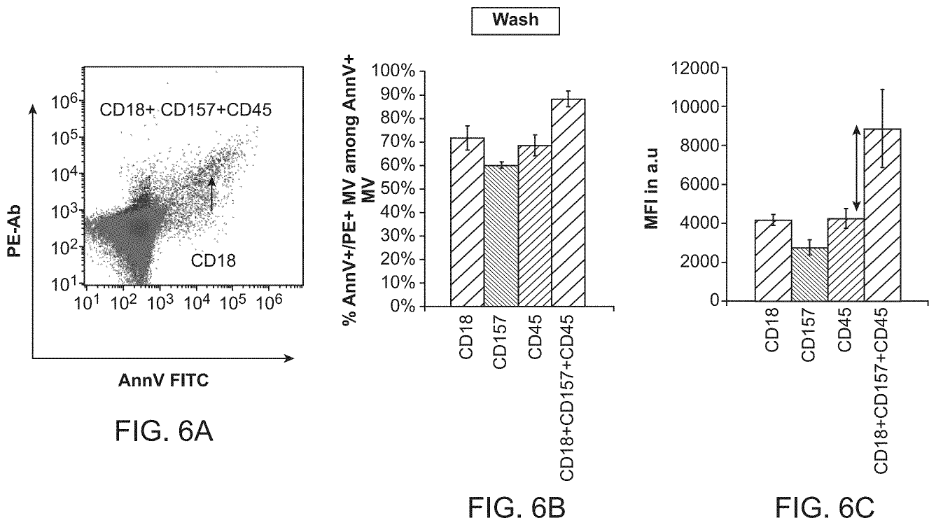

In this example, combinations of various antibodies that were previously identified as individually suitable for use to detect certain MV populations were used in the same assay. The purpose of the experiment was to determine if combination of the antibodies can improve sensitivity of the assay as compared to each of the antibodies used alone. In one experiment, CD18, CD157 and CD45, each labeled to PE and was previously shown to be able to detect myeloid-derived MVs (see Table 5), were combined and used to stain samples in the same assay. In this experiment, no wash step, i.e. no size exclusion chromatography was used to wash the stained mixture. FIGS. 5A-5C show under such no wash condition, the three-antibody combination detects higher percentages of MVs and produced slightly higher mean fluorescent intensity (MFI) than any of the three antibodies used alone. In another experiment, the same three-antibody combination was used to stain samples comprising MVs and the stained mixture were then washed with a SEC column before flow cytometry analysis. The results showed that the three-antibody combination detected higher percentages of MVs and produced significantly higher MFI (FIGS. 6A-6C).

### Example 5. Fluorescence Sensitive Cytometer for LMV Detection

The experiments in this example is to compare detection of MVs in the same sample by different flow cytometers: Gallios and CytoFLEX. As compared to Gallios, CytoFLEX has the advantages of being ultra-sensitive in detecting fluorescence, especially with the PE channel. In addition, CytoFLEX uses a yellow green laser that limits the signal spreading between the FITC and PE channels.

In one experiment, samples comprising MVs were stained with PE-labeled HLA DR antibody and FITC-labeled annexin V. The count of the double positive MVs count was more than 40% higher when analyzed using CytoFLEX as compared to using Gallios (FIG. 7A). In another experiment, samples comprising MVs were stained with PE-labeled CD11c antibody and FITC-labeled annexin V. When analyzed using Gallios, double positive populations were very low and difficult to be detected, and the MFI of PE channel was also very low. When analyzed using CytoFLEX, the MFI of the PE channel signal significantly increased and a significant amount of double positive population were detected. (FIG. 7B). FIG. 7C shows that statistically there were significant increases of MV counts when a sample comprising MVs was analyzed using CytoFLEX as compared to being analyzed using Gallios. The samples were stained with each one of HLA-DR, CD45, CD66c, CD14, CD31, CD18, CD157, CD11C, and CD15 antibodies.

1. A method of characterizing microvesicles comprising:
staining a sample including a microvesicle with a cocktail to form a stained microvesicle;
measuring the stained microvesicle by flow cytometry to obtain a signal set; and
identifying the stained microvesicle as a leukocyte-derived microvesicle when the signal set of the measured microvesicle is within a signal region.

2. The method of claim 1, wherein the cocktail includes labeled antibodies to at least one of HLA DR, CD11c, CD66c, CD18, and CD157.

3. The method of claim 2, wherein the cocktail comprises labeled annexin V.

4. The method of claim 2, wherein the cocktail comprises at least one of
i) a first group of labeled antibodies comprising an antibody to HLA DR and an antibody to CD11c, each labeled with a first label;
ii) a second group of labeled antibodies comprising an antibody to CD66c, labeled with a second label;
iii) a third group of labeled antibodies comprising an antibody to CD18 and an antibody to CD157, each labeled with a third label.

5. The method of claim 4, wherein second group further includes labeled antibodies to CD15, and wherein the third group further includes labeled antibodies to CD45.

6. The method of claim 4, wherein the labeled annexin V is labeled with a fourth label.

7. The method of claim 5, wherein the cocktail further includes antibodies to TIA-1 labeled with a fifth label.

8. The method of claim 5, wherein the step of identifying includes assigning the microvesicle as monocyte-derived when the signal set indicates binding of at least one antibody of the first group.

9. The method of claim 5, wherein the step of identifying includes assigning the microvesicle as granulocyte-derived when the signal set indicates binding of least one antibody of the second group.

10. The method of claim 5, wherein the step of identifying includes assigning the microvesicle as myeloid-derived when the signal set indicates binding of least one antibody of the third group.

11. The method of claim 7, wherein the step of identifying includes assigning the microvesicle as derived from an activated cell when the signal set indicates binding of antibody to TIA-1.

12. The method of claim 1, further including separating unbound material from the stained microvesicle.

13. The method of claim 12, wherein the step of separating unbound material includes washing the stained mixture on a size exclusion chromatography column loaded with a calcium-containing buffer.

14. A kit comprising a cocktail including labeled antibodies to at least one of HLA DR, CD11c, CD66c, CD18, and CD157.

15. A kit comprising a cocktail including at least one of:
a first group of labeled antibodies to HLA DR and CD11c;
a second group of labeled antibodies to CD66c;
a third group of labeled antibodies to CD18 and CD157; and
a fourth group of labeled antibodies to TIA-1.

16. The kit of claim 15, wherein the second group further includes labeled antibodies to CD15, and wherein the third group further includes labeled antibodies to CD45.

17. The kit of claim 15, wherein each of the antibodies in the first group is labeled with a first label, wherein each of the antibodies in the second group is labeled with a second label, wherein each of the antibodies in the third group is labeled with a third label, and wherein each of the antibodies in the fourth group is labeled with a fourth label.

18. The kit of claim 14, wherein the cocktail further includes annexin V labeled with a fifth label.

19. The kit of claim 14, further comprising an elution buffer.

20. A system for characterizing microvesicles comprising one or more processors and memory coupled to one or more processors, the memory encoded with a set of instructions configured to perform a method comprising:
obtaining measurements of a microvesicle stained with a cocktail from a flow cytometer, and
identifying the stained microvesicle as leukocyte-derived microvesicle when the signal set of the measured microvesicle is within a signal region.
